(19) United States
(12) Patent Application Publication
(10) Pub. No.: US 2014/0163270 A1
(43) Pub. Date: Jun. 12, 2014

(54) Hydroprocessing Of Biorenewable Feedstocks

(71) Applicants: Zen-Yu Chang, Houston, TX (US); C. David Roberts, Willoughby Hills, OH (US)

(72) Inventors: Zen-Yu Chang, Houston, TX (US); C. David Roberts, Willoughby Hills, OH (US)

(73) Assignee: THE LUBRIZOL CORPORATION, Wickliffe, OH (US)

(21) Appl. No.: 14/233,395

(22) PCT Filed: Jul. 23, 2012
(86) PCT No.: PCT/US2012/047789

§ 371 Date: Jan. 17, 2014

Related U.S. Application Data
(60) Provisional application No. 61/512,050, filed on Jul. 27, 2011.

(51) Int. Cl. C07C 1/22; C10L 10/04

(52) U.S. Cl. CPC C07C 1/22; C10L 10/04
USPC 585/240; 585/250

(57) ABSTRACT

The present invention provides an improved process for producing diesel boiling range fuel or fuel blending component from renewable feedstocks such as plant oils and greases. The improvement involved the addition of an organic polysulfide to the renewable feedstock before it enters the pre-reaction heating unit of the process resulting in reduced fouling in the pre-reaction heating unit. The invention also provides the use of such organic polysulfide in renewable feedstocks used in hydroprocessing equipment for reducing fouling in the pre-reaction heating units of such processes.

### FIELD OF THE INVENTION

The present invention provides an improved process for producing diesel boiling range fuel or fuel blending component from renewable feedstocks such as plant oils and greases. The improvement involved the addition of an organic polysulfide to the renewable feedstock before it enters the pre-reaction heating unit of the process resulting in reduced fouling in the pre-reaction heating unit. The invention also provides the use of such organic polysulfide in renewable feedstocks used in hydroprocessing equipment for reducing fouling in the pre-reaction heating units of such processes.

### BACKGROUND OF THE INVENTION

Environmental interests and an increasing worldwide demand for energy have encouraged energy producers to investigate renewable energy sources, including biofuels. Biofuel is obtained from biological material that is living or relatively recently lifeless, in contrast to fossil fuels (also referred to as mineral fuels) which are derived from ancient biological material. There is particularly interest in biofuels where, as in Europe, regulatory requirements have been or will be introduced that will require increased use of biofuels for motor vehicles, principally by blending with mineral fuels.

Biofuels are typically made from sugars, starches, vegetable oils, or animal fats using conventional technology from basic feedstocks, such as seeds, often referred to as bio-feeds. For example, wheat can provide starch for fermentation into bioethanol, while oil-containing seeds such as sunflower seeds provide vegetable oil that can be used in biodiesel.

The conventional approach for converting vegetable oils or other fatty acid derivatives into liquid fuels in the diesel boiling range is by a transesterification reaction with an alcohol, typically methanol, in the presence of catalysts, normally a base catalyst such as sodium hydroxide. The product obtained is typically a fatty acid alkyl ester, most commonly fatty acid methyl ester (known as FAME). While FAME has many desirable qualities, such as high cetane and its perceived environmental benefit, it has poor cold flow relative to mineral diesel because of its straight hydrocarbon chain. It also has lower stability because of the presence of ester moieties and unsaturated carbon-carbon bonds.

Hydrogenation methods are also known to convert vegetable oils or other fatty acid derivatives to hydrocarbon liquids in the diesel boiling range. These methods remove undesirable oxygen by hydrodeoxygenation to produce water, hydrodecarbonylation to produce CO, or hydrodecarboxylation to produce CO₂. In hydrodeoxygenation, unsaturated carbon-carbon bonds present in feed molecules are saturated (hydrogenated) before deoxygenation. Compared to transesterification, this type of hydrotreating has the practical advantage that it may be practiced in a refinery utilizing existing infrastructure. This reduces the need for investment and provides potential for operating on a scale that is more likely to be economical.

There are methods, developed by UOP (EcoFining) and Neste, which process triglycerides, such as found in vegetable oils, in a stand-alone manner. For instance, PCT Publication No. WO 2008/020048 describes a process for coprocessing triglycerides with heavy vacuum oil in single or multiple reactors, and partial hydrogenation of oxygenated hydrocarbon compounds such as glycerol is disclosed as being more desirable from the perspective of hydrogen consumption. PCT Publication No. WO 2008/012415 describes a process for the catalytic hydrotreatment of a feedstock derived from petroleum, of the gasoil type, in at least one fixed bed hydrotreatment reactor, wherein up to about 30% by weight of vegetable oils and/or animal fats are incorporated into the feedstock, and the reactor is operated in a single pass without recycle.

European Patent No. EP 1911735 describes co-hydrogenation of a carboxylic acid and/or derivative with a hydrocarbon stream from a refinery, as a retrofit. CoMo or NiMo catalysts are disclosed. It is stated that conditions are maintained in the reactor such that almost complete conversion of the carboxylic acid and/or ester is achieved, that is, greater than 90% conversion and preferably greater than 95% conversion. The product is described as suitable for use as or with a diesel fuel.

PCT Publication No. WO 2008/040973 describes a process, which is suitable as a retrofit, in which a mixed feed of carboxylic acid and/or derivatives including esters, and a refinery process stream, such as a diesel fuel, are hydrodeoxygenated or simultaneously hydrodesulfurized and hydrodeoxygenated. The catalyst may be Ni or Co in combination with Mo. The process produces a product which is described as suitable for use as diesel, gasoline or aviation fuel. It is stated that, under the described conditions, conversions of greater than 90% of the co-fed carboxylic acid and/or derivatives are typical and usually greater than 95% is achieved.

PCT Publication No. WO 2007/138254 describes a process in which in a first stage a hydrocarbon process stream, which may be a middle distillate, is hydrogenated and then fed with a carboxylic acid and/or ester to a second hydrogenation stage. The final product may be diesel fuel, and the benefits are said to be reduced exotherm, improved diesel yield, reduced fouling, reduced coking, and reduced residual olefins and/or heteroatoms. Mention is made of an alternative process in which an untreated hydrocarbon process stream is fed with the ester. Conditions in the second reactor are said to be the same as the first, and NiMo and CoMo are described as preferred catalysts for the first reactor. It is stated that conditions are maintained in the reactor such that almost complete conversion of the carboxylic acid and/or ester is achieved, that is greater than 90% conversion and preferably greater than 95% conversion.

Unites States Application 2009/0077865 describes means of reducing fouling and deposition formation in the reaction chamber of a hydroprocessing unit, but provides no teaching on controlling reducing fouling and deposition formation in the heat exchanges and/or furnaces used to pre-heat the feed stream before it enters the reaction chamber. The types of deposits involved and fouling involved in the reaction chamber are different from those at issue in the pre-reaction heating unit, as the reaction chamber is at a higher temperature, typically includes a catalyst bed that can themselves catalyze deposit formation, can themselves be fouled, and can have materials stripped from them by the process stream. These are different and unrelated problems to the issue of fouling in the pre-reaction heating unit.

All of these processes and approaches can be difficult to carry out over long periods of time due to fouling caused by deposit formation in the processing equipment, particularly in the pre-heating unit that the bio-feed stream typically passes through before entering the reaction unit. This pre-heating unit, or heat exchanger, brings the feed stream up to or near to the desired temperature for the reaction that is to take place in the reaction chamber unit.

When the pre-heating unit becomes fouled there is a reduction in the efficiency of the heat-exchange, as the fouling deposits act as an insulating layer, and can even begin to inhibit flow through the unit if the deposits are allowed to accumulate. The fouling of concern here includes fouling from: (i) pre-existing foulants present in the feed stream, such as insoluble inorganic debris including sand and corrosion scale, insoluble organic debris such as cellulose and lignin, marginally soluble components that become insoluble in areas of locally-high temperature including the heat exchanging surfaces of the pre-heating unit, such as asphaltenes in crude oil; and (ii) foulants formed by chemical reactions that take place in the pre-heating unit such as polymers formed by reactions at the locally-high temperature including the heat exchanging surfaces of the pre-heating unit, where such reaction may take place when the feed stream contains polymerizable components, trace metal components that act as catalysts, and dissolved oxygen.

When the pre-heating unit becomes fouled the entire process must be shut down in order to remove the deposits. This is a costly and time consuming activity that also reduces operation time for the equipment involved.

Therefore, there is a need for an improved hydrotreating process for biorenewable feedstocks, such as vegetable oils and animal fats, that reduces the amount of fouling seen in the pre-reaction heating unit of the process.

### SUMMARY OF THE INVENTION

The subject invention relates to a process for producing a hydrocarbon stream suitable for use as a fuel from a renewable feedstock, wherein said process comprises: feeding an oxygenate feed stream to a pre-reaction heating unit wherein an organic polysulfide is added to the oxygenate feed stream before it enters the pre-reaction heating unit in order to reduce fouling in said pre-reaction heating unit.

The subject invention relates to a process for producing a hydrocarbon stream suitable for use as a fuel from a renewable feedstock, wherein said process comprises: (a) feeding an oxygenate feed stream to a pre-reaction heating unit; (b) feeding said feed stream to a hydrotreatment reaction zone; (c) contacting the feed stream within the hydrotreatment reaction zone with a gas comprising hydrogen under hydrotreatment conditions; (d) removing a hydrotreated product stream; and (e) separating from the hydrotreated product stream a hydrocarbon stream suitable for use as fuel; wherein an organic polysulfide is added to the oxygenate feed stream before it enters the pre-reaction heating unit in order to reduce fouling in said pre-reaction heating unit. In some embodiments the hydrocarbon stream recovered after step e) is a diesel fuel.

Suitable oxygenate feed streams may be derived from a plant oil, an animal oil or fat, algae, waste oil, or a combination thereof. Specific examples include chicken fat and crude soy bean oil. The oxygenate feed stream may also be obtained by transesterification of C₈ to C₃₆ carboxylic esters with an alcohol in the presence of a base catalyst. Specific examples include fatty acid methyl esters.

The organic polysulfide may include a compound of the formula R—Sₓ—R, or a mixture of such compounds, where R is branched alkyl of 3 to 15 carbon atoms and x is either an integer between 1 and 8 or even 3 and 8. The organic polysulfide may be added to the feed stream in an amount of at least 100 or 1000 ppm based on the weight of said oxygenate feed stream.

The subject invention also relates to the use of an organic polysulfide in an oxygenate feed stream to reduce fouling in a pre-reaction heating unit of a hydroprocessing unit that converts said oxygenate feed stream to a hydrocarbon stream suitable for use as a fuel.

### DETAILED DESCRIPTION OF THE INVENTION

Various features and embodiments of the invention will be described below by way of non-limiting illustration.

### The Process

This invention generally relates to a process for hydroconversion of oxygenated hydrocarbon compounds. The hydroconversion process, or specific hydroprocessing unit suitable for use with the present invention is not overly limited so long as the unit employs a pre-reaction heating unit, for example a heat exchanger or furnace that heats the feed stream before it enters the reaction chamber, which may also be referred to as the hydrotreatment reaction zone.

The invention relates to a process for producing a hydrocarbon stream suitable for use as a fuel from a renewable feedstock, wherein said process comprises: feeding an oxygenate feed stream to a pre-reaction heating unit wherein an organic polysulfide is added to the oxygenate feed stream before it enters the pre-reaction heating unit in order to reduce fouling in said pre-reaction heating unit.

Processes for producing hydrocarbon streams suitable for use as fuels, where the feedstocks are carboxylic esters and similar renewable materials, may generally include the steps: (a) feeding an oxygenate feed stream to a pre-reaction heating unit; (b) feeding said feed stream to a hydrotreatment reaction zone; (c) contacting the feed stream within the hydrotreatment reaction zone with a gas comprising hydrogen under hydrotreatment conditions; (d) removing a hydrotreated product stream; and (e) separating from the hydrotreated product stream a hydrocarbon stream suitable for use as fuel.

In some embodiments the stream is reacted in the hydrotreatment reaction zone until no more than 86 wt % of the esters in the oxygenate feed stream are converted to hydrocarbons. In some embodiments the hydrotreated product stream obtained from the hydrotreatment reaction zone can be further hydrotreated in one or more additional hydrotreatment reaction zones by contacting the stream with hydrogen under hydrotreatment conditions until at least 90, 95 or even 99 wt % of the esters in the oxygenate feed stream are converted to hydrocarbons. The hydrotreated product stream can then be removed from the additional hydrotreatment reaction zone(s).

Thus, in some embodiments the invention comprises: (a) feeding an oxygenate feed stream to a pre-reaction heating unit; (b) feeding said feed stream to a first hydrotreatment reaction zone; (c) contacting the feed stream within the hydrotreatment reaction zone with a gas comprising hydrogen under hydrotreatment conditions until not more than 86% of the esters in the oxygenate feed stream are converted by hydrodeoxygenation to hydrocarbons; (d) removing from the first hydrotreatment reaction zone a first hydrotreated product stream; (e) contacting the hydrotreated product stream within at least a second hydrotreatment reaction zone with a gas comprising hydrogen under hydrotreatment conditions until at least 90, 95 or even 99 wt % of the esters in the oxygenate feed stream are converted to hydrocarbons; (f) removing from the second hydrotreatment reaction zone a second hydrotreated product stream; and (g) separating from the hydrotreated product stream a hydrocarbon stream suitable for use as fuel.

The hydrotreatment reaction may be carried out at temperatures in the range of from about 150 to about 430° C. and pressures of from about 0.1 to about 25 MPa or from 1 to 20 MPa or even 15 MPa. Where the hydrotreatment reaction is carried out in a single reaction zone, the temperature can range from about 200 to about 400° C., or from about 250 to about 380° C. However, in where there are two or more stages of hydrotreatment, the temperature in each reaction zone may be lower, as a milder hydrotreatment may be carried out. In such embodiments, the temperature can range from about 150 to about 300° C. or from about 200 to about 300° C. Additionally, in certain two stage hydrotreatment reaction zone embodiments, the temperature in the first reaction zone can be lower than the temperature in the second reaction zone.

The hydrogen used in any hydrotreatment process according to the invention may be a substantially pure, fresh feed, but it is also possible to use recycled hydrogen-containing feed from elsewhere in the process, or from the refinery, that may contain contamination from by-products, preferably such that the chemical nature and/or the concentration of the by-products in the hydrogen does not cause a significant reduction (e.g., not more than a 10% reduction, preferably not more than a 5% reduction) in the activity and/or lifetime of any catalyst to which the hydrogen is exposed. The hydrogen treat gas ratio can typically be in the range of about 50 Nm³/m; (about 300 scf/bbl) to about 1000 Nm³/m: (about 5900 scf/bbl). In certain embodiments, typically when relatively milder hydrotreatment conditions are desired, the hydrogen treat gas ratio can be from about 75 Nm³/m³ (about 450 scf/bbl) to about 300 Nm³/m³ (about 1800 scf/bbl) or from about 100 Nm³/m³ (about 600 scf/bbl) to about 250 Nm³/m³ (about 1500 scf/bbl). In other embodiments, typically when relatively harsher hydrotreatment conditions are desired, the hydrogen treat gas ratio can be from about 300 Nm³/m³ (about 1800 scf/bbl) to about 650 Nmr/m³ (about 3900 scf/bbl) or from about 350 Nmim³ (about 2100 scf/bbl) to about 550 Nm³/m³ (about 3300 scf/bbl).

The hydrotreatment step(s) may be catalyzed, and suitable catalysts include those comprising one or more Group VIII metals and one or more Group VIB metals, for example comprising Ni and/or Co and W and/or Mo, preferably comprising a combination of Ni and Mo, or Co and Mo, or a ternary combination such as Ni, Co, and Mo or such as Ni, Mo, and W. Each hydrotreatment catalyst is typically supported on an oxide such as alumina, silica, zirconia, titania, or a combination thereof, or another known support material such as carbon. Such catalysts are well known for use in hydrotreatment and hydrocracking.

A NiMo catalyst may be used to initiate olefin saturation at a lower inlet temperature. Most units are constrained by a maximum operating temperature, and large amounts of heat are released from treatment of biofeeds. Initiating olefin saturation at lower temperature with NiMo allows for longer cycle lengths (as the maximum temperature will be reached later) and/or permits processing of more biofeeds.

A CoMo catalyst may be used for lower hydrogen partial pressure desulfurization and to slow down the kinetics of biofeed treatment. Spreading the exotherm out throughout the process by having such a lower activity catalyst will reduce the number of hotspots (which decrease in efficiency of the unit, and potentially give rise to structural issues if near reactor walls). At high hydrogen partial pressures, the use of CoMo may also reduce the amount of methanation that occurs, which helps to reduce hydrogen consumption.

As used herein, the terms “CoMo” and “NiMo” refer to comprising oxides of molybdenum and either cobalt or nickel, respectively, as catalytic metals. Such catalysts may also optionally include supports and minor amounts of other materials such as promoters. By way of illustration, suitable hydrotreating catalysts are described, for example, in one or more of U.S. Pat. Nos. 6,156,695, 6,162,350, 6,299,760, 6,582,590, 6,712,955, 6,783,663, 6,863,803, 6,929,738, 7,229,548, 7,288,182, 7,410,924, and 7,544,632, U.S. Patent Application Publication Nos. 2005/0277545, 2006/0060502, 2007/0084754, and 2008/0132407, and International Publication Nos. WO 04/007646, WO 2007/084437, WO 2007/084438, WO 2007/084439, and WO 2007/084471.

A combination of catalysts may be used in the first or in the second (or subsequent) hydrotreatment reaction zones. These catalysts may be arranged in the form of a stacked bed. Alternatively, one catalyst may be used in first hydrotreatment reaction zone and a second catalyst in the second (or subsequent) hydrotreatment reaction zones. In a preferred arrangement the first hydrotreatment reaction zone comprises a stacked bed of NiMo catalyst, followed by a CoMo catalyst. The second reaction zone preferably comprises a CoMo catalyst. Nevertheless, in alternate arrangements stacked bed arrangements, the NiMo catalyst in the first hydrotreatment zone may be substituted with a catalyst containing Ni and W metals or a catalyst containing Ni, W, and Mo metals.

The hydrotreatment may be conducted at liquid hourly space velocities (LHSV) of from about 0.1 to about 10 hf⁻¹, for example from about 0.3 to about 5 hr⁻¹ or from about 0.5 to about 5 hr⁻¹. In the embodiments of the invention where there are two or more stages of hydrotreatment, the conditions in either or each reaction zone (or each reactor, where the reaction zones are in separate reactors) may be milder, and as indicated above this may be achieved by using lower temperatures. Alternatively or in addition, the LHSV may be increased to reduce severity. In such an embodiment, the LHSV is preferably from about 1 to about 5 hr⁻¹.

It is believed to be within the competence of one skilled in the art to select an appropriate catalyst, and then determine the specific conditions within the above-mentioned ranges under which the hydrotreatment according to the invention may be carried out, so that hydrodesulfurization of the hydrocarbon feed and conversion of the oxygenate feed to hydrocarbons can be achieved, e.g., without significant loss of hydrocarbons boiling in the diesel range due to unwanted hydrocracking.

Following hydrotreatment, whether in a single hydrotreatment step or in a sequence of two or more hydrotreatment steps, a hydrotreated product stream is recovered from the hydrotreatment and a hydrocarbon product stream suitable for use as fuel can then be separated from it. The hydrotreated product stream may be subjected to conventional separation processes to achieve this; for example, flash separation to remove light ends and gases, and fractionation to isolate hydrocarbons boiling in the diesel fuel range.

In addition, the hydrotreated product stream may be subjected to optional hydroisomerization over an isomerization catalyst to improve the properties of the final product, such as the cold flow properties.

In the embodiments of the invention where the hydrotreatment of an oxygenate feed stream comprising olefinic unsaturation and the hydrocarbon feed stream are carried out in two or more hydrotreatment reaction zones, the hydrotreatment is preferably conducted to split heat release between the two reaction zones. For example, in the first hydrotreatment reaction zone the olefins may be saturated, and the methyl or ethyl ester groups removed along with some oxygen removal, and then in the second hydrotreatment reactor the conversion to hydrocarbons suitable for use as fuel is completed. This enables each stage to be carried out under relatively milder conditions and with better control of heat release than would a single stage hydrotreatment to achieve similar hydrocarbon conversion.

The first hydrotreated product stream removed from the first hydrotreatment reaction zone may optionally be cooled before it is hydrotreated within the second hydrotreatment reaction zone using conventional means, such as heat exchangers or quench gas treatment. Heat recovered in this way may be used to preheat feed at other points in the process, such as the oxygenate feed to the first reaction zone.

A further option is to pass the first hydrotreated product stream through a separator to separate out any light ends, CO, CO₂, or water before it is passed into the second reaction zone. Such removal of the CO and water may improve catalyst activity and cycle length.

The recovered hydrocarbon product stream may be used as fuel, such as diesel fuel, heating oil, or jet fuel, either alone or combined with other suitable streams. A preferred use of the hydrocarbon product stream is as diesel fuel and it may be sent to the diesel fuel pool. It may also be subjected to further convention treatments, including the addition of additives to enhance the performance, e.g., as a diesel fuel.

This invention extends to a fuel, such as diesel fuel, heating oil, or jet fuel, when prepared by the process as described herein.

In one embodiment, the recovered product hydrocarbon stream can comprise at least 90, 93, or 95 wt % saturated hydrocarbons typically up to about 98, 99, 99.5 or even 99.9 wt %, and less than 1, 0.5, 0.2, or 0.1 wt % ester-containing compounds. In further embodiments the recovered product hydrocarbon stream may include less than 500, 200 or even 100 weight ppm (wppm) ester-containing compounds. In still other embodiments recovered product hydrocarbon stream may contain no more than 100, 200, or 500 wppb, or 1, 2, 5, or 10 wppm ester-containing compounds If any; no more than 1, 0.5, 0.2, 0.1 wt % or no more than 500, 200, 100, 75, 50, or even 25 wppm acid-containing compounds If any; not more than 10 wppm sulfur-containing compounds, based on the total weight of the product hydrocarbon stream. In this embodiment, the product hydrocarbon stream can be used as, and/or can be used as a blend component in combination with one or more other hydrocarbon streams, to form a diesel fuel, a jet fuel, a heating oil, or a portion of a distillate pool.

In another embodiment, where there are at least first and second hydrotreatment reaction zones, the partially converted first hydrotreated product stream from step (b)(ii) can comprise from about 30 wt % to about 60 wt % of compounds containing only hydrogen and carbon atoms, at least about 4 wt % trans-esterified (i.e., containing the alkyl group from the alcohol, preferably methyl) ester-containing compounds, at least about 2 wt % acid-containing compounds that are fully saturated, and at least about 0.3 wt % alkyl alcohols, based on the total weight of the partially converted first hydrotreated product stream.

In some embodiments the renewable feedstock is co-fed with petroleum derived feedstocks in a standard or modified hydroconversion process. Mixtures of oxygenated compounds, such as those found in bio-oils derived from pyrolysis or liquefaction, are also included in the definition of biomass-derived oxygenated compound. In some embodiments the process of the invention uses only the renewable feedstock and no petroleum derived feedstocks are co-fed into the process.

### The Pre-Heating Unit

As noted above, the invention related to a process for producing a hydrocarbon stream useful as diesel fuel from renewable feedstocks where the process uses a pre-reaction heating unit that heats the renewable feedstock stream before it enters the reaction zone.

The pre-reaction heating unit is typically one or more heat exchangers or furnaces positioned before the hydrotreatment reaction zone. The pre-reaction heating unit brings the feed stream up to the desired temperature before it enters the reaction zone. This desired temperature may be the desired reaction temperature, or it may be just below the desired reaction temperature.

As noted above, fouling and deposit formation in the pre-reaction heating unit is a serious problem for hydroprocessing unit. The deposits that form in the pre-reaction heating unit tend to be different from those that are a concern in the hydrotreatment reaction zone. Deposits in the pre-reaction heating unit tend to be more related to the renewable feedstock stream, including impurities and debris in the stream itself. In contrast, deposits in the hydrotreatment reaction zone tend to be more related to undesired reaction byproducts. Furthermore the impact of fouling and deposits in these two regions of the process are very different. In the pre-reaction heating unit, fouling and deposits can impact the heat exchange, thus causing the feed stream to come into the reaction zone cooler than desired and/or requiring more energy and cost to get the feed stream up to the desired temperature. In the reaction zone, deposit control and fouling are almost solely focused on the catalysts, ensuring the catalyst is available to facilitate the desired reactions, and is not itself fouled. In other words, the deposits and fouling concerns in the pre-reaction heating unit are different from the deposits and fouling concerns in the reaction zone.

The pre-reaction heating unit may, in some embodiments, bring the feed stream up to the desired reaction temperature, including any of the reaction temperatures described above. In other embodiments the pre-reaction heating unit may bring the feed stream up to a temperature 5, 10 or even 15° C. below the desired reaction temperature, including any of the reaction temperatures described above.

### The Feed Stream

The present invention relates to a process for producing a hydrocarbon stream useful as diesel fuel from renewable feedstocks such as those originating from plants or animals. This renewable feedstock may be referred to as an oxygenate stream or simply as the renewable or bio renewable feed stream.

The term renewable feedstock is meant to include feedstocks other than those obtained from petroleum crude oil. Another term that has been used to describe this class of feedstock is bio-renewable fats and oils. The renewable feedstocks that can be used in the present invention include any of those which comprise glycerides and free fatty acids (FFA) as well as other fatty acid esters. Most of the glycerides will be triglycerides, but monoglycerides and diglycerides may be present and processed as well.

Examples of these renewable feedstocks include, but are not limited to, canola oil, corn oil, soy oils, rapeseed oil, soybean oil, colza oil, tall oil, sunflower oil, hempseed oil, olive oil, linseed oil, coconut oil, castor oil, peanut oil, palm oil, mustard oil, jatropha oil, tallow, yellow and brown greases, lard, train oil, fats in milk, fish oil, algal oil, sewage sludge, wood pulp, derivative of wood pulp, and the like. Additional examples of renewable feedstocks include non-edible vegetable oils from the group comprising Jatropha curcas (Ratanjoy, Wild Castor, Jangli Erandi), Madhuca indica (Mohuwa), Pongamia pinnata (Karanji Honge), and Azadiracta indicia (Neem).

The triglycerides and FFAs of the typical vegetable or animal fat contain aliphatic hydrocarbon chains in their structure which have about 8 to about 24 carbon atoms with a majority of the fats and oils containing aliphatic hydrocarbon chains with 16 and 18 carbon atoms.

Mixtures or co-feeds of renewable feedstocks and petroleum derived hydrocarbons may also be used as the feedstock. Other feedstock components which may be used, especially as a co-feed component in combination with the above listed feedstocks, include spent motor oils and industrial lubricants, used paraffin waxes, liquids derived from the gasification of coal, biomass, or natural gas followed by a downstream liquefaction step such as Fischer-Tropsch technology, liquids derived from depolymerization, thermal or chemical, of waste plastics such as polypropylene, high density polyethylene, and low density polyethylene; and other synthetic oils generated as byproducts from petrochemical and chemical processes. Mixtures of the above feedstocks may also be used as co-feed components. One advantage of using a co-feed component is the transformation of what has been considered to be a waste product from a petroleum based or other process into a valuable co-feed component to the current process.

Renewable feedstocks that can be used in the present invention may contain a variety of impurities. For example, tall oil is a byproduct of the wood processing industry and tall oil contains esters and rosin acids in addition to FFAs. Rosin acids are cyclic carboxylic acids. The renewable feedstocks may also contain contaminants such as alkali metals, e.g. sodium and potassium, phosphorus as well as solids, water and detergents. An optional first step is to remove as much of these contaminants as possible. One possible pretreatment step involves contacting the renewable feedstock with an ion-exchange resin in a pretreatment zone at pretreatment conditions.

In some embodiments the oxygenate feed stream is derived from biomass, and is preferably derived from plant oils such as rapeseed oil, palm oil, peanut oil, canola oil, sunflower oil, tall oil, corn oil, soybean oil, olive oil, jatropha oil, jojoba oil, and the like, and combinations thereof. It may additionally or alternately be derived from animal oils and fats, such as fish oil, lard, tallow, chicken fat, milk products, and the like, and combinations thereof, and/or from algae. Waste oils such as used cooking oils can also be used.

A typical feed stream contains alkyl (preferably methyl and/or ethyl, for example methyl) esters of carboxylic acids such as methyl esters of saturated acids (typically having from 8 to 36 carbons attached to the carboxylate carbon, preferably from 10 to 26 carbons, for example from 14 to 22 carbons), which may contain one more unsaturated carbon-carbon bonds. In some embodiments the feed stream includes: methyl esters of C₁₈ saturated acids, methyl esters of C₁₈ acids with 1 olefin bond; methyl esters of C₁₈ acids with 2 olefin bonds; methyl esters of C₁₈ acids with 3 olefin bonds; or methyl esters of C₂₀ saturated acids.

As used herein, the phrase “alkyl ester”, with reference to esters of carboxylic acids should be understood to mean a straight or branched hydrocarbon having from 1 to 24, 1 to 18, 1 to 12, or even 1 to 8 carbon atoms attached via an ester bond to a carboxylate moiety. For clarity, though a preferred alkyl ester of a carboxylic acid includes fatty acid esters such as FAME, there is no requirement that the alkyl esters of carboxylic acids be characterized as “fatty acid” esters in order to be useful in the invention.

The oxygenate feed stream may be derived from biomass by a transesterification reaction with an appropriate alcohol, that is a C₁ to C₂₄ alcohol, in the presence of catalysts, normally a base catalyst such as sodium hydroxide, to obtain a fatty acid alkyl ester (e.g., where the alkyl group is a methyl and/or ethyl group). The oxygenate feed stream may contain esters of carboxylic acids which are saturated or unsaturated, with unsaturated esters containing one or more, typically one, two or three, olefinic groups per molecule. Examples of unsaturated esters include esters of oleic, linoleic, palmitic, and stearic acid. A preferred oxygenate feed stream comprises one or more methyl or ethyl esters of carboxylic acids.

An oxygenate feed stream comprising one or more methyl or ethyl esters of carboxylic acids may be derived from biomass by a transesterification reaction with the appropriate alcohol, that is methanol and/or ethanol. In some embodiments the oxygenate feed stream comprises fatty acid methyl ester (FAME), although, where a lower net greenhouse gas emissions effect process is of increased importance, processing of fatty acid ethyl esters (FAEE) can be advantageous (due to the use of ethanol instead of methanol as a transesterification agent).

The renewable feed stream may include oxygenated hydrocarbon compounds that have been produced via the liquefaction of a solid biomass material. In a specific embodiment the oxygenated hydrocarbon compounds are produced via a mild hydrothermal conversion process, such as described in EP 061135646, filed on May 5, 2006. In an alternate specific embodiment the oxygenated hydrocarbon compounds are produced via a mild pyrolysis process, such as described in EP 061135679, filed on May 5, 2006.

The renewable feed stream may be mixed with an inorganic material, for example as a result of the process by which they were obtained. In particular, solid biomass may have been treated with a particulate inorganic material in a process such as described in co-pending application EP 061135810, filed May 5, 2006. These materials may subsequently be liquefied in the process of EP 061135646 or that of EP 061135679, cited herein above. The resulting liquid products contain the inorganic particles. It is not necessary to remove the inorganic particles from the oxygenated hydrocarbon compounds prior to the use of these compounds in the process of the present invention. To the contrary, it may be advantageous to leave the inorganic particles in the oxygenated hydrocarbon feed, in particular if the inorganic material is a catalytically active material. In the alternative the inorganic material may be used as a catalyst carrier.

Similarly, the oxygenated hydrocarbon compounds may have been obtained by liquefaction of a biomass material comprising an organic fiber, as disclosed in co-pending application EP 06117217.7, filed Jul. 14, 2006. In this case the oxygenated hydrocarbon compounds may contain organic fibers. It may be advantageous to leave these fibers in the reaction feed, as they may have catalytic activity. The fibers may also be used as a catalyst carrier, for example by bringing the fibers into contact with a metal.

In some embodiments the oxygenate feed stream is derived from a plant oil, an animal oil or fat, algae, waste oil, or a combination thereof. For example, the feed stream may be chicken fat or soybean oil, including crude (unrefined) soybean oil.

In other embodiments the oxygenate feed stream is obtained by the transesterification of C₈ to C₃₆ carboxylic esters with an alcohol in the presence of a base catalyst. In some of these embodiments the oxygenate feed stream comprises fatty acid methyl esters.

The oxygenate feed stream can include canola oil, corn oil, rapeseed oil, soybean oil, colza oil, tall oil, sunflower oil, hempseed oil, olive oil, linseed oil, coconut oil, castor oil, peanut oil, palm oil, mustard oil, cottonseed oil, inedible tallow, yellow and brown greases, lard, train oil, fats in milk, fish oil, algal oil, sewage sludge, ratanjoy oil, wild castor oil, jangli oil erandi oil, mohuwa oil, karanji honge oil, neem oil, and mixtures thereof.

In still further embodiments, any of the oxygenate feed streams discussed above may further include a co-feed component. Suitable co-feed components include spent motor oils, spent industrial lubricants, used paraffin waxes, liquids derived from the gasification of coal followed by a downstream liquefaction step, liquids derived from the gasification of biomass followed by a downstream liquefaction step, liquids derived from the gasification of natural gas followed by a downstream liquefaction step, liquids derived from depolymerization of waste plastics, synthetic oils, and mixtures thereof.

### The Organic Polysulfide

The polysulfides of interest include those with the formula R—Sₓ—R where R is a linear or branched alkyl of 2 to 15 or 3 to 15 carbon atoms and x is either an integer between 1 and 8 or 2 to 8 or even 3 to 8. In some embodiments a mixture of polysulfides is used.

SulfrZol™ 54, available from the Lubrizol Corporation, is an example of a suitable polysulfide, which may be described by the formula R—Sₓ—R where x can be 4 for about 30-50 number percent of the molecules or 3 to 6 for about 80-95 number percent of the molecules. Trace amounts of molecules where x is 1, 2, 7, or 8 may also be present.

In some embodiments each R in the formula above would be a linear or branched alkyl of 2 to 10 or 3 to 10 carbon atoms, and in some embodiments each R would be a t-butyl group. In some embodiments at least 50%, on a molar basis, of the polysulfides have R groups that are t-butyl groups.

The amount of polysulfide added to the feed stream would depend on the specific properties of the feed stream being used. In some embodiments one could adjust the amount of polysulfide added in order to control the deposit formation in the pre-heating unit. The adjusted amount, found to control deposit formation, may be considered the effective amount for the specific feed stream being used. In other embodiments the polysulfide could be used in amounts such that it adds at least 10 ppm or 50 ppm of sulfur to the feed stream, or so it adds from about 20 to about 300 ppm or even from 50 to 250 ppm of sulfur. In some embodiments the polysulfide could be used in amounts such that it adds about 50 and 400 ppm or even 75 to about 300 ppm of sulfur to the feed stream. In still other embodiments, the polysulfide itself may be added so that it is present at least 100, or even at least 200, 300, 400 or even 1000 ppm in the feed stream, on a weight basis. In some embodiments the polysulfide itself is added such that it is present at 100 to 1000, 200 to 800, 300 to 700, 400 to 600, or even 450 to 550, or 500 ppm in the feed stream, on a weight basis.

### The Use of an Organic Polysulfide to Reduce Pre-Heating Unit Fouling

The subject invention also relates to the use of an organic polysulfide in an oxygenate feed stream to reduce fouling in a pre-reaction heating unit of a hydroprocessing unit that converts said oxygenate feed stream to a hydrocarbon stream suitable for use as a fuel. The organic polysulfide may be any of the materials described above and may be used in any of the amounts provided. In some embodiments, the use of the organic polysulfide is solely for the reduction of fouling and/or deposit formation in the pre-heating unit, and is not added to reduce fouling and/or deposit formation in the reaction chamber, to reduce fouling and/or deposit formation on the catalysts used in the reaction chamber, or to protect and/or restore any sulfur in the catalysts used in the reaction chamber.

As used herein, the term “hydrocarbyl substituent” or “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include: hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form a ring); substituted hydrocarbon substituents, that is, substituents containing non-hydrocarbon groups which, in the context of this invention, do not alter the predominantly hydrocarbon nature of the substituent (e.g., halo (especially chloro and fluoro), hydroxy, alkoxy, mercapto, alkylmercapto, nitro, nitroso, and sulfoxy); hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in the hydrocarbyl group. As used herein, the term “hydrocarbonyl group” or “hydrocarbonyl substituent” means a hydrocarbyl group containing a carbonyl group.

It is known that some of the materials described above may interact in the final formulation, so that the components of the final formulation may be different from those that are initially added. For instance, metal ions (of, e.g., a detergent) can migrate to other acidic or anionic sites of other molecules. The products formed thereby, including the products formed upon employing the composition of the present invention in its intended use, may not be susceptible of easy description. Nevertheless, all such modifications and reaction products are included within the scope of the present invention; the present invention encompasses the composition prepared by admixing the components described above.

### EXAMPLES

This invention is illustrated by the following examples that are merely for the purpose of illustration and are not to be regarded as limiting the scope of the invention or the manner in which it can be practiced. Unless specifically indicated otherwise, parts and percentages are given by weight.

The examples described below are evaluated using a laboratory-scale thermal fouling test with a hot liquid process simulator (HLPS). The laboratory thermal fouling test is an accelerated test designed to simulate the fouling or coking problems experienced in refinery or petrochemical processes, including hydroprocessing units. The test operating temperature is usually higher than those seen in a plant in order to accelerate the simulation and reproduce and evaluate fouling problems in a reasonable time. For this testing all examples were evaluated using a 6 hour run time. The tests can be done under inert atmosphere such as nitrogen or under air, with air considered to be a harsher test condition. The tests can be done by one-pass through or by a recycling mode. Normally, the tests are done in one-pass mode but in some cases recycling mode is used in order to further accelerate the testing as recycle mode is considered a harsher test condition.

The test procedure includes passing the renewable feedstock through a resistance heated tube-in-shell heat exchanger, which simulates the pre-reaction heating unit. The system is pressurized during the test to prevent the fluid from vaporizing in the heat exchanger. The test proceeds by holding constant the heat exchanger internal surface temperature while monitoring the change in the liquid outlet temperature. If fouling occurs (i.e. a fouling deposit builds up on the surface of the heat exchanger heating tube) a decrease in the fluid outlet temperature occurs which corresponds to fouling characteristics of the fluid being tested. The degree of change in the temperatures can be used to calculate an overall effectiveness of the system, that is, the amount of antifouling prevented compared to the baseline system. Under the test conditions here, a higher effectiveness indicates more of the fouling expected (that seen in the baseline) has been avoided. This antifouling effectiveness is calculated as a percentage value relative is a baseline, using the following formula: Percent Effectiveness=(ΔT_{BASE}−ΔT_{EX})/ΔT_{BASE} where the ΔT_{BASE} is the change in outlet temperature seen in the baseline test and ΔT_{EX} is the change in outlet temperature seen in the test run using the additive material.

### Example Set 1

Example Set 1 uses a chicken fat renewable feed stock. The material used in each example is the same chicken fat material, however Example 2 is treated with one additive (Additive A) and Examples 3 and 4 are treated with a different additive (Additive B), while Example 1 is an non-additized baseline. Example 2 contains 395 ppm of a dialkyl disulfide (Additive A), Example 3 contains 500 ppm of a mixture of polysulfides including di-tert-butyl polysulfides (Additive B) and Example 4 contains 185 ppm of Additive B.

The feed stock is tested using the laboratory-scale thermal fouling test described above using a nitrogen atmosphere in recycle mode for a 6 hour test run. The results collected are summarized in the table below:

TABLE 1

| | Example 1 | Example 2 | Example 3 | Example 4 |
|---|---|---|---|---|
| | (baseline) | (395 ppm A) | (500 ppm B) | (185 ppm B) |
| Heat Exchanger | 271 | 271 | 271 | 271 |
| Temp (° C.) | | | | |
| Atmosphere | Nitrogen | Nitrogen | Nitrogen | Nitrogen |
| Operation Mode | Recycle | Recycle | Recycle | Recycle |
| Test Time (hrs) | 6.0 | 6.0 | 6.0 | 6.0 |
| Heating Oil Flow | 6.0 | 6.0 | 6.0 | 6.0 |
| Rate (cc/min) | | | | |
| Temperature | 22 | 17 | 13 | 16 |
| Change (ΔT, ° C.) | | | | |
| Antifouling | NA | 23% | 41% | 27% |
| Effectiveness (%) | | | | |

The results show that the antifouling effectiveness of renewable feed stocks such as chicken fat can be improved by the addition of an organic polysulfide. The results also show that Additive B is more effective than Additive A at reducing fouling.

### Example Set 2

Example Set 2 uses a crude soy bean oil feed stock. The material used in each example is the same crude soy bean oil material, however Example 5 is treated with 500 ppm of a mixture of polysulfides including di-tert-butyl polysulfides (Additive B).

The feed stock is tested using the laboratory-scale thermal fouling test described above using an air atmosphere in recycle mode for a 6 hour test run. The results collected are summarized in the table below:

TABLE 2

| | Example 4 | Example 5 |
|---|---|---|
| | (baseline) | (500 ppm B) |
| Heat Exchanger Temp (° C.) | 215 | 215 |
| Atmosphere | Air | Air |
| Operation Mode | Recycle | Recycle |
| Test Time (hrs) | 6.0 | 6.0 |
| Heating Oil Flow Rate (cc/min) | 6.0 | 6.0 |
| Temperature Change (ΔT, ° C.) | 19 | 3 |
| Antifouling Effectiveness (%) | NA | 84% |

The results show that the antifouling effectiveness of renewable feed stocks such as a crude soybean oil can be improved by the addition of an organic polysulfide.

Each of the documents referred to above is incorporated herein by reference. Except in the Examples, or where otherwise explicitly indicated, all numerical quantities in this description specifying amounts of materials, reaction conditions, molecular weights, number of carbon atoms, and the like, are to be understood as modified by the word “about.” Except where otherwise indicated, all numerical quantities in the description specifying amounts or ratios of materials are on a weight basis. Unless otherwise indicated, each chemical or composition referred to herein should be interpreted as being a commercial grade material which may contain the isomers, by-products, derivatives, and other such materials which are normally understood to be present in the commercial grade. However, the amount of each chemical component is presented exclusive of any solvent or diluent oil, which may be customarily present in the commercial material, unless otherwise indicated. It is to be understood that the upper and lower amount, range, and ratio limits set forth herein may be independently combined. Similarly, the ranges and amounts for each element of the invention can be used together with ranges or amounts for any of the other elements. As used herein, the expression “consisting essentially of” permits the inclusion of substances that do not materially affect the basic and novel characteristics of the composition under consideration.

We claim:

1. A process for producing a hydrocarbon stream suitable for use as a fuel from a renewable feedstock, wherein said process comprises: feeding an oxygenate feed stream to a pre-reaction heating unit wherein an organic polysulfide is added to the oxygenate feed stream before it enters the pre-reaction heating unit in order to reduce fouling in said pre-reaction heating unit.

2. A process for producing a hydrocarbon stream suitable for use as a fuel from a renewable feedstock, wherein said process comprises:
a) feeding an oxygenate feed stream to a pre-reaction heating unit;
b) feeding said feed stream to a hydrotreatment reaction zone;
c) contacting the feed stream within the hydrotreatment reaction zone with a gas comprising hydrogen under hydrotreatment conditions;
d) removing a hydrotreated product stream; and
e) separating from the hydrotreated product stream a hydrocarbon stream suitable for use as fuel;
wherein an organic polysulfide is added to the oxygenate feed stream before it enters the pre-reaction heating unit in order to reduce fouling in said pre-reaction heating unit.

3. The process of claim 1 wherein said oxygenate feed stream is derived from a plant oil, an animal oil or fat, algae, waste oil, or a combination thereof.

4. The process of claim 1 wherein said oxygenate feed stream is obtained by transesterification of C₈ to C₃₆ carboxylic esters with an alcohol in the presence of a base catalyst.

5. The process of claim 1 wherein said oxygenate feed stream comprises fatty acid methyl esters.

6. The process of claim 1 wherein said hydrocarbon stream recovered after step e) is a diesel fuel.

7. The process of claim 1 further comprising feeding a petroleum derived feed stream in the reaction zone with the oxygenate feed stream.

8. The process of claim 1 wherein the oxygenate feed stream comprises at least one component selected from the group consisting of canola oil, corn oil, soy oil, rapeseed oil, soybean oil, colza oil, tall oil, sunflower oil, hempseed oil, olive oil, linseed oil, coconut oil, castor oil, peanut oil, palm oil, mustard oil, cottonseed oil, inedible tallow, yellow and brown greases, lard, train oil, fats in milk, fish oil, algal oil, sewage sludge, ratanjoy oil, wild castor oil, jangli oil erandi oil, mohuwa oil, karanji honge oil, neem oil, and mixtures thereof.

9. The process of claim 1 wherein the oxygenate feed stream further comprises at least one co-feed component selected from the group consisting of spent motor oils, spent industrial lubricants, used paraffin waxes, liquids derived from the gasification of coal followed by a downstream liquefaction step, liquids derived from the gasification of biomass followed by a downstream liquefaction step, liquids derived from the gasification of natural gas followed by a downstream liquefaction step, liquids derived from depolymerization of waste plastics, synthetic oils, and mixtures thereof.

10. The process of claim 1 wherein said organic polysulfide comprises a compound of the formula R—Sₓ—R where R is branched alkyl of 3 to 15 carbon atoms and x is an integer between 1 and 8.

11. The process of claim 1 wherein said organic polysulfide comprises a compound of the formula R—Sₓ—R where R is branched alkyl of 3 to 15 carbon atoms and x is an integer between 3 and 8.

12. The process of claim 1 wherein said organic polysulfide comprises a mixture of compounds each having the formula R—Sₓ—R where R is branched alkyl of 3 to 15 carbon atoms and x is an integer between 1 and 8.

13. The process of claim 1 wherein R is a branched alkyl group containing from 3 to 10 carbon atoms.

14. The process of claim 1 wherein at least 50% of the R groups of said organic polysulfide are tert-butyl groups.

15. The process of claim 1 wherein said organic polysulfide is added in an amount of at least 100 or 1000 ppm based on the weight of said oxygenate feed stream.

16. The use of an organic polysulfide in an oxygenate feed stream to reduce fouling in a pre-reaction heating unit of a hydroprocessing unit that converts said oxygenate feed stream to a hydrocarbon stream suitable for use as a fuel.
